(19) United States
(12) Patent Application Publication
(10) Pub. No.: US 2014/0160429 A1
(43) Pub. Date: Jun. 12, 2014

(54) OPHTHALMIC IMAGING APPARATUS, CONTROL METHOD OF OPHTHALMIC IMAGING APPARATUS AND STORAGE MEDIUM

(71) Applicant: CANON KABUSHIKI KAISHA, Tokyo (JP)

(72) Inventors: Yoshitaka Nakano, Kawasaki-shi (JP); Satoshi Aikawa, Yokohama-shi (JP); Hideyuki Ohban, Saitama-shi (JP); Yohei Saito, Chigasaki-shi (JP)

(73) Assignee: CANON KABUSHIKI KAISHA, Tokyo (JP)

(21) Appl. No.: 14/093,613

(22) Filed: Dec. 2, 2013

(30) Foreign Application Priority Data
Dec. 12, 2012 (JP) 2012-271776

(51) Int. Cl. A61B 3/12; A61B 3/14

(52) U.S. Cl. CPC A61B 3/12; A61B 3/14
USPC 351/206; 351/246

(57) ABSTRACT

An apparatus comprises: a projection unit arranged in an illumination optical system for projecting illumination light onto a fundus of an eye and to project a focus index onto the eye; a focus lens arranged in a light-receiving optical system for guiding reflected light from the fundus to an image sensor and to focus the image sensor on the fundus; a first unit to detect an approximate focus position using the focus index in a first mode; a second unit to detect a focus position in a second mode by evaluating a luminance-contrast of a fundus image formed on the image sensor based on the approximate focus position; and a control unit to control positions of the focus lens and the projection unit in association with each other in the first mode, and control them independently in the second mode.

### BACKGROUND OF THE INVENTION

1. Field of the Invention

The present invention relates to an ophthalmic imaging apparatus, a control method of the ophthalmic imaging apparatus, and a storage medium and, more particularly, to an ophthalmic imaging apparatus for observing or capturing the fundus of an eye to be examined, like a fundus camera used in an ophthalmic clinic, mass health screening, or the like, a control method of the ophthalmic imaging apparatus, and a storage medium.

2. Description of the Related Art

To easily obtain focus on the fundus of an eye to be examined, a conventional fundus camera is known to project divided focus indices onto the pupil of the eye to be examined, observe the images via the focus lens of the observation imaging system, and observe the positional relationship of the focus index images, thereby obtaining focus. It is also known to capture the projected focus indices and automatically focus based on the positional relationship of the focus index images.

However, if the focus index images are simply set in a predetermined positional relationship (lined up in a straight line), a focusing error may occur due to the influence of the aberration of the eye optical system caused by astigmatism or the like, resulting in a fundus image out of focus.

Japanese Patent Laid-Open No. 2009-268772 proposes an ophthalmic imaging apparatus that sets the focus index images in a predetermined positional relationship and then performs auto focusing using the contrast of the focus index images, thereby performing accurate auto focusing. In the ophthalmic imaging apparatus described in Japanese Patent Laid-Open No. 2009-268772, auto focusing is performed for the focus index images, thereby reducing the influence of the aberration of the eye optical system caused by the astigmatism or the like of the eye to be examined for the focus index image projection unit. However, the influence of the aberration of the eye optical system caused by the astigmatism or the like of the eye to be examined still remains for portions on the fundus where the focus index images are not projected.

To the contrary, Japanese Patent Laid-Open No. 2011-50532 proposes an ophthalmic imaging apparatus that detects the line of sight and the left and right eyes so as to predict a specific portion (for example, medium and large blood vessels) on the fundus and decide a region to perform focus detection, thereby performing auto focusing using the contrast of the specific portion. In the ophthalmic imaging apparatus described in Japanese Patent Laid-Open No. 2011-50532, auto focusing is performed for the specific portion on the fundus, thereby reducing the influence of the aberration of the eye optical system caused by the astigmatism or the like of the eye to be examined for the specific portion on the fundus.

In auto focusing by the ophthalmic imaging apparatus described in Japanese Patent Laid-Open No. 2011-50532, however, it is necessary to drive the focus lens throughout the focus range of the fundus image of the eye to be examined and detect the contrast of the specific portion on the fundus. Hence, a time is needed until the focus position is detected, and it is therefore impossible to detect the correct focus position because of small involuntary eye movement during fixation or blink of the eye to be examined.

To solve these problems, focus detection may be performed using a first focus detection unit that approximately detects the focus position of an eye to be examined using focus indices captured by an image sensor and a second focus detection unit that detects, using the approximately detected focus position as a reference, the focus position of the eye to be examined by detecting the contrast of the fundus image of the eye to be examined captured by the image sensor. In this case, automatic control is done while associating the position of the focus lens and the position of the focus index projection unit with each other such that the image sensor and the focus index projection unit that projects the focus indices are optically placed at conjugate positions.

Since the first focus detection unit using the focus indices can perform the focus detection at a high speed, the influence of small involuntary movement or blink of the eye to be examined can largely be reduced. In addition, the influence of the aberration of the eye optical system caused by astigmatism or the like of the eye to be examined can be reduced by the second focus detection unit that detects the focus position on the fundus of the eye.

However, when controlling while associating the position of the focus lens with the position of the focus index projection unit, if a difference is generated between the first focus position detection result and the second focus position detection result due to the individual difference and the like, the focus index images may be unable to line up after auto focusing. This may mislead the operator into determining that no focus state is obtained.

### SUMMARY OF THE INVENTION

In consideration of the above-described problems, the present invention provides a technique of reducing the possibility of misleading the operator into determining that no focus state is obtained upon observing focus index images when executing a focusing operation using a plurality of focus position detection units.

According to one aspect of the present invention, there is provided an ophthalmic imaging apparatus comprising: a focus index projection unit arranged in an illumination optical system for projecting illumination light onto a fundus of an eye to be examined and configured to project a focus index onto the eye to be examined; a focus lens arranged in a light receiving optical system for guiding reflected light from the fundus to an image sensor and configured to focus the image sensor on the fundus; a first focus detection unit configured to detect an approximate focus position using the focus index in a first control mode; a second focus detection unit configured to detect a focus position in a second control mode by evaluating a luminance contrast of a fundus image formed on the image sensor based on the approximate focus position; and a control unit configured to control a position of the focus lens and a position of the focus index projection unit in association with each other in the first control mode, and control the position of the focus lens and the position of the focus index projection unit independently in the second control mode.

According to one aspect of the present invention, there is provided a control method of an ophthalmic imaging apparatus including a focus index projection unit arranged in an illumination optical system for projecting illumination light onto a fundus of an eye to be examined and configured to project a focus index onto the eye to be examined, and a focus lens arranged in a light receiving optical system for guiding reflected light from the fundus to an image sensor and configured to focus the image sensor on the fundus, the method comprising: a first focus detection step of detecting an approximate focus position using the focus index in a first control mode; a second focus detection step of detecting a focus position in a second control mode by evaluating a luminance contrast of a fundus image formed on the image sensor based on the approximate focus position; and a control step of controlling a position of the focus lens and a position of the focus index projection unit in association with each other in the first control mode, and control the position of the focus lens and the position of the focus index projection unit independently in the second control mode.

Further features of the present invention will be apparent from the following description of exemplary embodiments with reference to the attached drawings.

### BRIEF DESCRIPTION OF THE DRAWINGS

FIG. 1 is a view showing an example of the arrangement of an ophthalmic imaging apparatus according to an embodiment of the present invention;

FIG. 2 is an explanatory view of details of a focus detection unit 30 according to the embodiment of the present invention;

FIG. 3 is an enlarged view of a fundus image displayed on a monitor 15 according to the embodiment of the present invention;

FIG. 4 is a view showing the relationship between the contrast value and the state of focus index images in a region A 301 according to the embodiment of the present invention;

FIG. 5 is a view for explaining focus position detection by a first focus detection unit 202 according to the embodiment of the present invention;

FIG. 6 is a graph showing the principle of contrast detection according to the embodiment of the present invention;

FIG. 7 is a flowchart showing the procedure of control mode switching processing executed by the ophthalmic imaging apparatus according to the embodiment of the present invention;

FIG. 8 is a flowchart showing the procedure of first focus position detection processing executed by the ophthalmic imaging apparatus according to the embodiment of the present invention; and

FIG. 9 is a flowchart showing the procedure of second focus position detection processing executed by the ophthalmic imaging apparatus according to the embodiment of the present invention.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

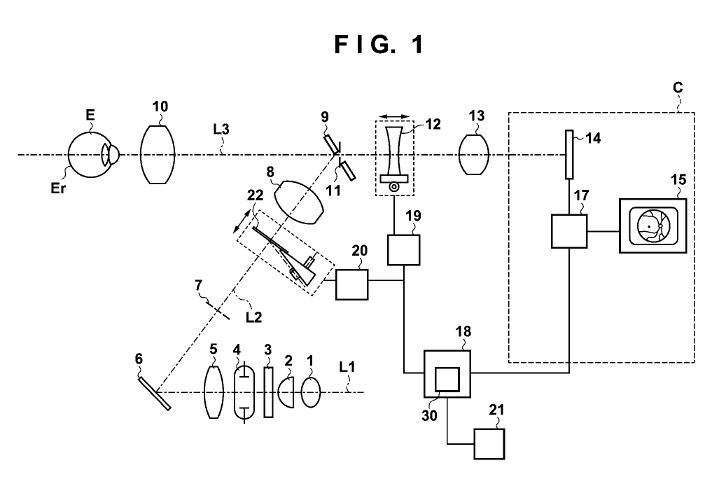

An example of the arrangement of an ophthalmic imaging apparatus according to this embodiment will be described with reference to FIG. 1. An observation light source 1 such as a halogen lamp that emits fixed light (illumination light), a condenser lens 2, a filter 3 that passes infrared light and blocks visible light, an imaging light source 4 such as an electronic flash, a lens 5, and a mirror 6 are arranged on an optical axis L1. A ring aperture 7 having a ring-shaped opening, a relay lens 8, and a perforated mirror 9 having an opening at the center are sequentially arranged on an optical axis L2 of the illumination optical system in the reflecting direction of the mirror 6.

An objective lens 10 is arranged on an optical axis L3 of the light receiving optical system in the reflecting direction of the perforated mirror 9 and faces an eye E to be examined. An imaging aperture 11 is provided in the opening of the perforated mirror 9. In addition, a focus lens 12 that adjusts focus by moving on the optical axis L3 of the light receiving optical system for guiding reflected light from a fundus Er of the eye E to be examined to an image sensor 14, and an imaging lens 13 are arranged. The image sensor 14 having both a moving image observation function and a still image capturing function is arranged in an imaging camera C ahead of the imaging lens 13.

The focus lens 12 focuses the image sensor 14 on the fundus. The output from the image sensor 14 is sent to an image processing unit 17 connected to the image sensor 14. The output from the image processing unit 17 is sent to a control unit 18 connected to the image processing unit 17. The image processing unit 17 displays, on a monitor 15, an observation image captured by the image sensor 14.

On the other hand, a focus index projection unit 22 is arranged between the ring aperture 7 and the relay lens 8 on the optical axis L2 of the illumination optical system. The focus index projection unit 22 and the focus lens 12 are driven by a focus index driving unit 20 and a focus lens driving unit 19, respectively, under the control of the control unit 18.

In a manual focusing mode, the control unit 18 can control the focus lens driving unit 19 and the focus index driving unit 20 in accordance with an operation input from an operation input unit 21. At this time, the operator performs the operation such that the focus index projection unit 22 and the image sensor 14 optically have a conjugate relationship.

In an auto focusing mode, the control unit 18 controls the focus lens driving unit 19 and the focus index driving unit 20 in a first control mode and a second control mode to be described later in accordance with processing of a first focus detection unit 202 and a second focus detection unit 203 to be described later. The control unit 18 also executes control of light amount adjustment, on/off, and the like of the observation light source 1 and control of light amount adjustment, on/off, and the like of the imaging light source 4.

In the above-described arrangement, the control unit 18 turns on the observation light source 1. An illumination light beam emitted by the observation light source 1 is condensed by the condenser lens 2. The filter 3 removes visible light and passes only infrared light. The infrared light passes through the imaging light source 4 such as an electronic flash and the lens 5 and is reflected by the mirror 6. The infrared light reflected by the mirror 6 is converted into a ring-shaped light beam by the ring aperture 7 and deflected in the direction of the optical axis L3 by the relay lens 8 and the perforated mirror 9. Then, the light illuminates the fundus Er via the objective lens 10.

The light beam that has reached the fundus Er is reflected and scattered by the fundus Er. The light then exits from the eye E to be examined, passes through the objective lens 10, the imaging aperture 11, the focus lens 12, and the imaging lens 13, and forms an image on the image sensor 14. The control unit 18 controls the image processing unit 17 and displays the fundus image captured by the image sensor 14 on the monitor 15.

The examiner performs fine adjustment to align the eye E to be examined with the optical unit while observing the fundus image displayed on the monitor 15. After focus adjustment, the examiner performs imaging by pressing an imaging switch (not shown). The ophthalmic imaging apparatus according to this embodiment has an auto focusing function of automatically executing the focus adjustment.

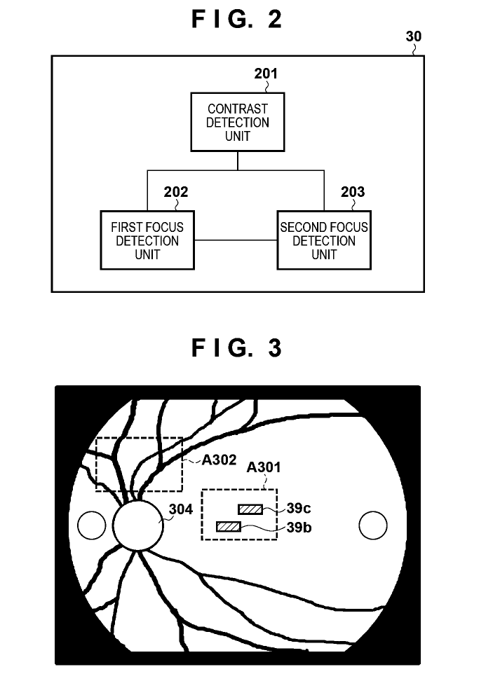

The detailed arrangement of a focus detection unit 30 according to this embodiment will be described next with reference to FIG. 2. The focus detection unit 30 includes a contrast detection unit 201, the first focus detection unit 202, and the second focus detection unit 203 which are used for focusing. The contrast detection unit 201 is connected to the image sensor 14 via the image processing unit 17 and also connected to the first focus detection unit 202 and the second focus detection unit 203. The first focus detection unit 202 and the second focus detection unit 203 are connected to each other to synchronize the start of focus detection. That is, both the first focus detection unit 202 and the second focus detection unit 203 are configured to perform focus detection using the contrast detection unit 201.

The focus detection position and range where the first focus detection unit 202 and the second focus detection unit 203 perform detection will be described next with reference to FIG. 3. FIG. 3 is an enlarged view of a fundus image displayed on the monitor 15. A region A 301 indicates the focus detection position and range of the first focus detection unit 202. A region A 302 indicates the focus detection position and range of the second focus detection unit 203. The region A 301 includes focus index images including a focus index image 39b and a focus index image 39c. The region A 302 includes medium and large blood vessels on the retina.

Note that in this embodiment, the second focus detection unit 203 detects the medium and large blood vessels on the retina. However, the second focus detection unit 203 may detect, for example, a papillary portion 304 at a position and range where the focus index images to be detected by the first focus detection unit 202 are not displayed. As described above with reference to FIGS. 2 and 3, in this embodiment, the first focus detection unit 202 and the second focus detection unit 203 are configured to perform focus detection by contrast detection of the focus index images and focus detection by contrast detection of the fundus portion different from the focus index images, respectively.

<Function of First Focus Detection Unit 202>

The function of the first focus detection unit 202 will be described first. In detection processing of the first focus detection unit 202, control of the focus index driving unit 20 is executed in the first control mode in which the position of the focus lens 12 and the position of the focus index projection unit 22 are controlled in association with each other. In the first control mode, when one of the focus lens 12 and the focus index projection unit 22 is driven, the other is also driven synchronously and moves its position. That is, the focus index driving unit 20 automatically controls the position of the focus lens 12 and the position of the focus index projection unit 22 such that the focus index projection unit 22 and the image sensor 14 are optically placed at conjugate positions.

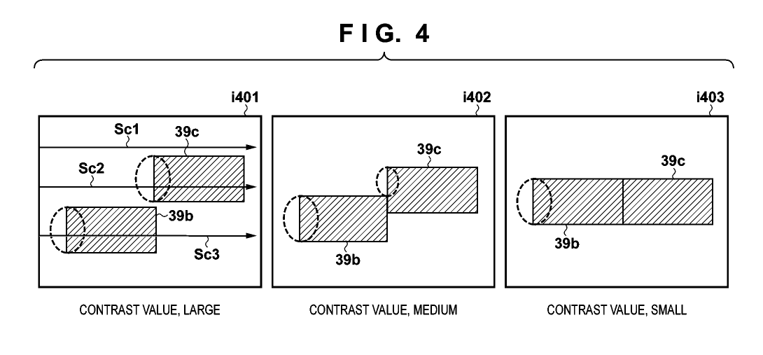

FIG. 4 shows the relationship between the contrast value and the state of the focus index images in the region A 301 according to this embodiment. Images i401 to i403 indicate the states of the focus index images when the focus index projection unit 22 is driven in the region A 301 shown in FIG. 3. In the images i401 to i403, each of the focus index images 39b and 39c can be observed.

Scan lines Sc1, Sc2, and Sc3 in the image i401 represent the state of scan to evaluate the contrast of the image by the contrast detection unit 201. In this case, “contrast” indicates the luminance difference between adjacent pixels, and “contrast value” is the maximum luminance difference value in the luminance data of the scan lines. The arrows of the scan lines Sc1, Sc2, and Sc3 indicate the scan directions. Lines corresponding to the number of vertical pixels from the upper portion to the lower portion are scanned in the horizontal direction in accordance with the size of the image i401.

The contrast value of the whole image i401 is calculated by scanning lines corresponding to the number of vertical pixels from the upper portion to the lower portion and totaling the contrast values calculated for the respective lines. Hence, portions of the images i401 to i403 indicated by the dotted lines are calculated as the contrast values of the images.

Hence, the contrast value of the image i401 is obtained as the sum of the value of one focus index image 39b and the value of one focus index image 39c indicated by the dotted lines. Similarly, the contrast value of the image i402 is obtained as the sum of the value of one focus index image 39b and the value of ½ of the focus index image 39c. The contrast value of the image i403 is obtained as the value of one focus index image 39b. That is, the calculated contrast value becomes small in the order of the image i401, the image i402, and the image i403. The images i401 to i403 are the images of the region A 301 in FIG. 3 and therefore have the same position and size.

In all of the images i401 to i403, the focus index images 39b and 39c are assumed to have the same luminance, and the portions other than the focus index images are assumed to have the same luminance for the sake of simplicity. In the actually observed fundus image, however, the focus index images and the portions other than the focus index images have various luminance distributions depending on how the focus index images look or noise in the portions other than the focus index images. However, since the focus index images 39b and 39c are projected in a high contrast, the difference between the luminance of the focus index images and the luminance of the portions other than the focus index images is dominant in the contrast value.

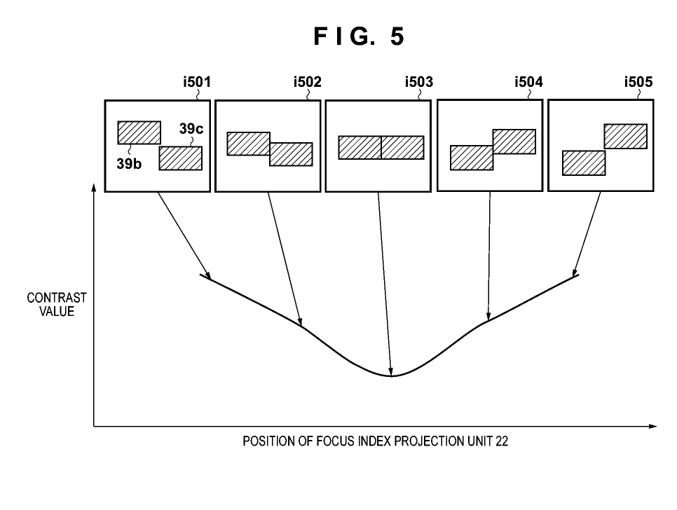

First focus position detection processing will be described with reference to FIG. 5, in which the first focus detection unit 202 detects a position where the shift between the focus index images is minimized using the difference in the magnitude of the contrast value depending on the positions of the focus index images 39b and 39c.

Images i501 to i505 indicate the states of the focus index images in the region A 301 shown in FIG. 3, as in FIG. 4. The images i501 to i505 represent the focus index images when the focus index projection unit 22 is driven throughout its movable range. The states of the focus index images 39b and 39c can be observed.

FIG. 5 shows the transition of the contrast value with respect to the position of the focus index projection unit 22, which is represented by a line that connects the contrast values obtained from the images i501 to i505.

As shown in FIG. 5, the image i503 in which the shift between the focus index images 39b and 39c is minimized has the smallest contrast value. That is, the position of the focus index projection unit 22 corresponding to the image i503 matches the position where the shift between the focus index images is minimized. That is, it is only necessary to detect a position corresponding to the minimum one of the contrast values obtained from the images i501 to i505.

<Function of Second Focus Detection Unit 203>

The function of the second focus detection unit 203 will be described next. In detection processing of the second focus detection unit 203, control of the focus index driving unit 20 is executed in the second control mode in which the position of the focus lens 12 and the position of the focus index projection unit 22 are controlled independently. More specifically, in the second control mode, control of the focus index driving unit 20 is executed such that the focus index projection unit 22 stops at a position according to the detection result of the first focus detection unit 202. For this reason, the focus index images 39b and 39c continuously look as if they were in focus.

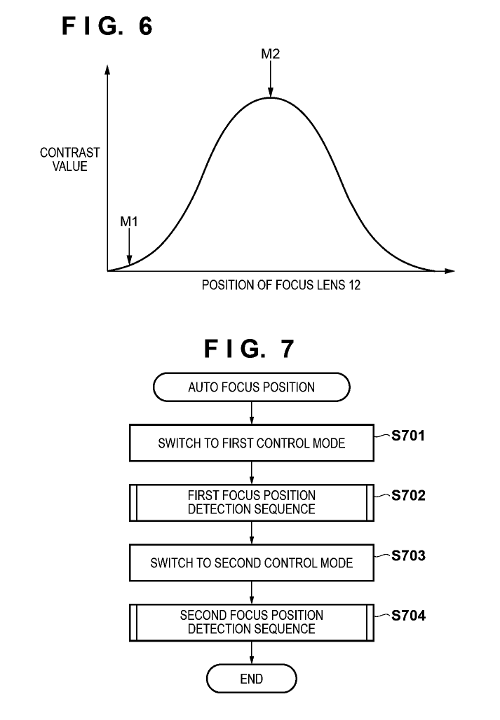

Second focus position detection processing of the second focus detection unit 203 will be described next with reference to FIG. 6. The range of focus detection executed by the second focus detection unit 203 is the range including the medium and large blood vessels on the retina in the region A 302 shown in FIG. 3. FIG. 6 shows the transition of the contrast value with respect to the position of the focus lens 12 driven by the focus lens driving unit 19. The contrast value calculation method is the same as the calculation method described with reference to FIG. 4. In FIG. 4, the difference between the luminance of the portions other than the focus index images and the luminance of the left side surfaces of the focus index images 39b and 39c is calculated as the contrast value of the entire image. In FIG. 6, however, the difference between the luminance of portions other than the medium and large blood vessels on the retina and the luminance of the ends of the medium and large blood vessels is calculated as the contrast value.

As shown in FIG. 6, the contrast value is maximized at a focus position M2. The contrast value is small at a position M1 where the lens is largely out of focus. The focus position M2 of the focus lens 12 driven by the focus lens driving unit 19 is the position where the fundus image displayed on the monitor 15 can be observed most clearly, and also matches the position of the focus lens 12 where the fundus image displayed on the monitor 15 after imaging becomes clearest. For this reason, in this embodiment, it is possible to perform focus detection using this principle of detecting the contrast value without the influence of the aberration of the eye to be examined.

The procedure of control mode switching processing (auto focusing sequence) executed by the ophthalmic imaging apparatus according to this embodiment will be described next with reference to the flowchart of FIG. 7.

When auto focusing starts, the control unit 18 switches control of the focus lens driving unit 19 and the focus index driving unit 20 to the first control mode in step S701.

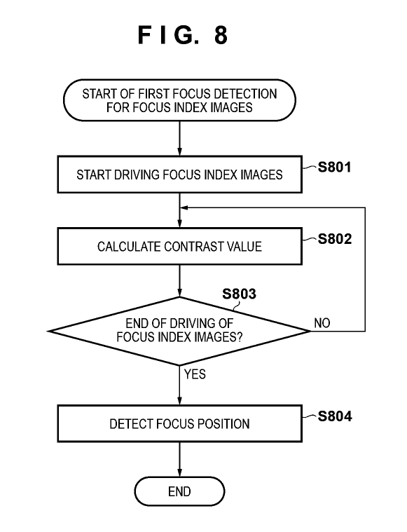

In step S702, the first focus detection unit 202 executes approximate focus position detection processing (first focus position detection processing) using the focus indices. Details will be described later with reference to the flowchart of FIG. 8.

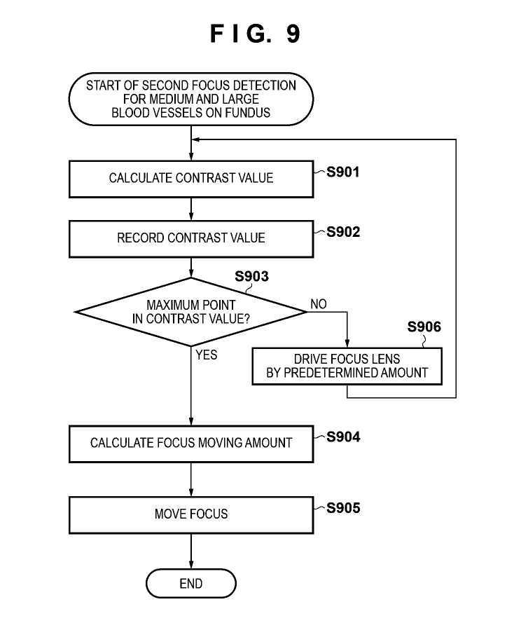

In step S703, the control unit 18 switches control of the focus lens driving unit 19 and the focus index driving unit 20 to the second control mode. In step S704, the second focus detection unit 203 executes focus position detection processing (second focus position detection processing) using the luminance contrast of the fundus image using the approximate focus position detected in step S702 as a reference. Details will be described later with reference to the flowchart of FIG. 9. The auto focusing thus ends.

The procedure of first focus position detection processing of the first focus detection unit 202 will be described with reference to the flowchart of FIG. 8. When the focus indices are projected onto the fundus, focus detection for the focus index images starts.

In step S801, the control unit 18 controls the focus index driving unit 20 in the first control mode and moves the focus index projection unit 22. Driving of the focus index images thus starts.

In step S802, the contrast detection unit 201 performs contrast value detection described with reference to FIGS. 4 and 5 for the region A 301 in FIG. 3. In step S803, the control unit 18 drives the focus index projection unit 22. The control unit 18 detects whether the focus index projection unit 22 has reached the end position in a direction opposite to the start position, thereby determining whether to end driving of the focus index projection unit 22. If the focus index projection unit 22 has reached the end position, and it is determined to end driving of the focus index projection unit 22 (YES in step S803), the process advances to step S804. On the other hand, if the focus index projection unit 22 has not reached the end position, and it is determined to continue driving of the focus index projection unit 22 (NO in step S803), the process returns to step S802.

In step S804, the first focus detection unit 202 detects the approximate focus position where the shift between the focus index images 39b and 39c is minimized based on the contrast values recorded in step S802, as described with reference to FIGS. 4 and 5. The position of the focus lens 12 at which the focus index projection unit 22 and the image sensor 14 optically have a conjugate relationship can be controlled by the control unit 18. The focus lens 12 is moved to a position corresponding to the position where the shift between the focus index images is minimized.

The procedure of second focus position detection processing of the second focus detection unit 203 will be described next with reference to the flowchart of FIG. 9. In this embodiment, focus detection processing for the medium and large blood vessels on the fundus starts based on the position of the focus lens moved in step S804 of FIG. 8.

More specifically, the focus lens position when starting focus detection for the medium and large blood vessels on the fundus need only fall within the range including the focus positions of the medium and large blood vessels on the fundus using the position where the shift between the focus index images is minimized as a reference. For example, when the relationship between the position where the shift between the focus index images is minimized and the position of the medium and large blood vessels on the fundus is defined as ±3 diopter, the focus lens position need only fall within the range of ±3 diopter.

In step S901, the contrast detection unit 201 performs contrast value calculation for the medium and large blood vessels on the fundus. In step S902, the second focus detection unit 203 records the contrast value calculated in step S901.

In step S903, the second focus detection unit 203 determines whether the contrast value recorded in step S902 includes the maximum point corresponding to the position M2 as shown in FIG. 6. Upon determining that the maximum point is included (YES in step S903), the process advances to step S904. On the other hand, upon determining that the maximum point is not included (NO in step S903), the process advances to step S906.

In step S904, the second focus detection unit 203 calculates the moving amount of the focus lens 12 from the current position to the position where the maximum point is detected. In step S905, the focus lens driving unit 19 drives the focus lens 12 in accordance with the moving amount of the focus lens 12 calculated in step S904, and moves the position of the focus lens 12 to a position where the contrast value is maximized.

In step S906, the focus lens driving unit 19 drives the focus lens 12 by a predetermined amount. After that, the process returns to step S901. The processes of steps S901, S902, S903, and S906 are repeated until the maximum point is detected.

When only the first focus detection using the focus indices is performed, it may be impossible to obtain focus on the fundus Er even if the focus index images 39b and 39c are lined up in a straight line due to a large optical aberration caused by the spherical aberration or astigmatism of the eye E to be examined. However, when the second focus detection is performed in the above-described way, focus adjustment can be performed in accordance with the aberration even if there is an individual difference in the aberration.

In the processing shown in FIG. 9, the focus index projection unit 22 controlled in the second control mode stops at the position obtained as the result of first focus position detection. For this reason, when the process of step S905 has ended, the focus index images look as if they were lined up as in the image i503 shown in FIG. 5 independently of the second focus position detection result. Hence, the operator can easily visually recognize the focus state.

### Second Embodiment

In the first embodiment, an arrangement has been described which stops the focus index projection unit 22 at the position of the detection result of the first focus detection unit 202 in the second control mode for controlling the focus index driving unit 20 during second focus position detection processing. In the second embodiment, however, in the second control mode for controlling a focus index driving unit 20 during second focus position detection processing, control is performed to arbitrarily drive a focus index projection unit 22 on an optical axis L2. In synchronism with the end of the second focus position detection processing, the focus index projection unit 22 is stopped at the position of the detection result of a first focus detection unit 202.

In the second control mode according to this embodiment, the focus index driving unit 20 is controlled such that the focus index projection unit 22 finely oscillates within a predetermined range with respect to the position of the detection result of the first focus detection unit 202 as a center during second focus position detection processing. The operator can observe focus index images 39b and 39c moving, and can therefore recognize that focus position detection has not ended yet.

In synchronism with the end of the second focus position detection processing, the focus index driving unit 20 is stopped at the position of the detection result of the first focus detection unit 202. For this reason, the operator can recognize that the focus index images indicate the focus state.

As described above, according to this embodiment, the operator can easily judge whether auto focusing has ended by visually checking the fine movement state/stop state of the focus index images. Note that not the fine oscillation but another control such as large oscillation may be used as long as the operator can recognize that focus detection has not ended.

### Third Embodiment

In the first embodiment, an arrangement has been described which stops the focus index projection unit 22 at the position of the detection result of the first focus detection unit 202 in the second control mode for controlling the focus index driving unit 20 during second focus position detection processing. In the third embodiment, however, a focus index projection unit 22 is retracted out of the optical axis (out of an optical axis L2) when performing second focus position detection processing in the second control mode.

At the start of second focus position detection processing, the focus index projection unit 22 is retracted out of the optical axis L2. For this reason, focus index images 39b and 39c are not projected onto the fundus observation image, and the operator cannot visually recognize them.

Hence, even if the result of first focus position detection processing and the result of second focus position detection processing are different due to an individual difference or the like, the operator can be prevented from becoming confused as the auto focusing ends while keeping the focus index images projected in a non-focus state.

As described above, according to this embodiment, the focus index images are made invisible for the operator, thereby avoiding confusion.

According to the present invention, it is possible to reduce the possibility of misleading the operator into determining that no focus state is obtained upon observing focus index images when executing a focusing operation using a plurality of focus position detection units.

### OTHER EMBODIMENTS

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-271776, filed on Dec. 12, 2012, which is hereby incorporated by reference herein in its entirety.

What is claimed is:

1. An ophthalmic imaging apparatus comprising:
a focus index projection unit arranged in an illumination optical system for projecting illumination light onto a fundus of an eye to be examined and configured to project a focus index onto the eye to be examined;
a focus lens arranged in a light receiving optical system for guiding reflected light from the fundus to an image sensor and configured to focus the image sensor on the fundus;
a first focus detection unit configured to detect an approximate focus position using the focus index in a first control mode;
a second focus detection unit configured to detect a focus position in a second control mode by evaluating a luminance contrast of a fundus image formed on the image sensor based on the approximate focus position; and
a control unit configured to control a position of said focus lens and a position of said focus index projection unit in association with each other in the first control mode, and control the position of said focus lens and the position of said focus index projection unit independently in the second control mode.

2. The apparatus according to claim 1, wherein in the second control mode, said control unit controls to stop said focus index projection unit at a position of a detection result of said first focus detection unit.

3. The apparatus according to claim 1, wherein in the second control mode, said control unit controls to make said focus index projection unit oscillate within a predetermined range with respect to a position of a detection result of said first focus detection unit as a center, and after an end of focus position detection by said second focus detection unit, controls to stop said focus index projection unit at the position of the detection result of said first focus detection unit.

4. The apparatus according to claim 1, wherein in the second control mode, said control unit controls to retract said focus index projection unit out of an optical axis of the illumination optical system.

5. A control method of an ophthalmic imaging apparatus including a focus index projection unit arranged in an illumination optical system for projecting illumination light onto a fundus of an eye to be examined and configured to project a focus index onto the eye to be examined, and a focus lens arranged in a light receiving optical system for guiding reflected light from the fundus to an image sensor and configured to focus the image sensor on the fundus, the method comprising:
a first focus detection step of detecting an approximate focus position using the focus index in a first control mode;
a second focus detection step of detecting a focus position in a second control mode by evaluating a luminance contrast of a fundus image formed on the image sensor based on the approximate focus position; and
a control step of controlling a position of the focus lens and a position of the focus index projection unit in association with each other in the first control mode, and control the position of the focus lens and the position of the focus index projection unit independently in the second control mode.

6. A non-transitory computer-readable storage medium storing a computer program that causes a computer to execute each step of a control method of an ophthalmic imaging apparatus of claim 5.
